(19) United States
(12) Patent Application Publication
(10) Pub. No.: US 2009/0088686 A1
(43) Pub. Date: Apr. 2, 2009

(54) Method of locating vessel puncture access sites via tattoo or permanent marking

(76) Inventor: Harold D. Cook, Dana Point, CA (US)

Correspondence Address: STETINA BRUNDA GARRED & BRUCKER, 75 ENTERPRISE, SUITE 250, ALISO VIEJO, CA, 92656, US

(21) Appl. No.: 11/904,369

(22) Filed: Sep. 27, 2007

(51) Int. Cl. A61M 5/42

(52) U.S. Cl. 604/116

(57) ABSTRACT

A method of marking the boundaries of at least one vessel with a vessel access marking for locating a vessel puncture access site to facilitate the introduction or withdrawal of fluid from the vein in connection with a medical procedure, the method comprising the steps of: designating an anatomical portion of an individual on which the medical procedure is performed; locating a vessel along the anatomical portion of the individual; applying a marking material to designate the boundaries of the vein, the marking material defining a region for vessel puncture access; cleansing an area proximate the vessel access marking and accessing the region of vessel puncture access using the vessel access marking as a visual reference.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The invention relates to the application of a marking material onto the skin for visual identification of at least one access site to facilitate diagnostic or therapeutic medical procedures. More specifically, the invention relates to a method of locating and marking the boundaries of at least one vessel, nonlimitedly exemplified by a vein, capillary or artery, using an ink, a dye or other type of marking material to designate a region of venous puncture access and performing a diagnostic or therapeutic procedure using the marking as a guide.

Many diagnostic and therapeutic procedures involve the penetration of the skin and entry into the body tissues, vessels and other subcutaneous structures to deliver medications, intravenous fluids, or to obtain blood and biopsy specimens. In particular, venous blood sampling is a procedure routinely performed in a doctor's office, laboratory or in the hospital that involves removal of blood from the human body. Often referred to as venipuncture, this procedure involves obtaining a sample of blood from a vessel of a human or any other animal for the purpose of testing for the presence or absence of a possibly suffered medical condition. For the purposes of this application, vessel is used interchangeably with vein, which is a type of vessel. It should be noted that a vessel may include, without limitation, any of the following: arteries, arterioles, capillaries, venules and veins and the method described herein is equally applicable to any type of vessel.

An individual may undergo venous blood sampling for any reason. For example, certain medical conditions, such as those relating to the kidneys and the liver, require the frequent draw of blood from an individual. For example, diabetes is a chronic disease that affects the ability of the body to produce or respond to insulin, which is the hormone that allows glucose to enter the cells of the body and be stored or used for energy. Many diabetic individuals not only require insulin injections, but they must also monitor and manage blood glucose levels. The required close and frequent monitoring of blood glucose translates to frequent blood draw, sometimes up to several times a day. Although some monitoring is done through self-monitoring, which involves the testing by the individual of blood obtained by a finger prick, diabetic individuals must still engage in physician-monitored blood testing. Similarly, individuals undergoing various types of treatment for cancer require frequent blood tests. For example, individuals receiving treatment for cancer must monitor their blood cell counts, including white blood cells, red blood cells and platelets. Such, blood cell counts must be monitored regularly to determine if any treatment has caused damage to the bone marrow. Further continuing advances in cancer treatment have resulted in an increased demand for venipuncture to effectively monitor certain cancer associated factors to evaluate treatment success. Outside of any defined medical need, an individual may elect to undergo venipuncture on a regular basis for the purpose of blood donation.

When performing any medical procedure that involves accessing the fragile veins of the body, it is important to select an access site carefully to preserve the integrity of the veins. For example, when performing venous blood sampling, a phlebotomist must first find a suitable vein from which to draw blood on an appropriate anatomical portion of the individual. This is typically done using a technique that includes visual inspection followed by palpation of the area to obtain a location from which blood is to be drawn. Once the skin is palpated and the location is selected, the phlebotomist must cleanse the area with antiseptic before beginning the procedure. If the phlebotomist does not remember the exact location, the area should not be repalpated, as repalpation would render the cleansed area recontaminated. Thus, the phlebotomist must depend on visual clues and memory to relocate the selected vein.

The integrity of the blood specimen depends in large part on appropriate vein selection in addition to a good technique of the phlebotomist. Thus, venipuncture should be done properly on the first attempt. If a venipuncture is unsuccessful on the first attempt, the phlebotomist usually can make one more attempt. However, only two unsuccessful attempts are generally permitted per individual to prevent complications and injury to the veins. Frequent blood draw may be inconvenient, difficult and also painful under certain conditions making a successful venipuncture attempt near impossible. For example, when the drawee is dehydrated, a suitable vein may be difficult to locate because the veins are constricted and located away from the surface of the skin. In the elderly, the veins become more fragile due to the decrease in the amount of connective tissue in the outer layer of the veins, which also limits the number of suitable veins. Also, while there is a general location of a particular vessel in the average individual, may individuals have veins that are not exactly in an expected location which may lead to unanticipated complications and difficulties.

Further, great care must be taken to avoid certain veins. For example, thready veins bleed profusely if they are punctured and flatten out if too severe of a vacuum is exerted on the vein. Next, superficial veins are tiny veins that appear on the surface of the arm and especially in the elderly. These veins are not appropriate for the draw of blood because they do not carry an adequate blood supply. Finally, thrombosed veins are veins that have been subjected to overuse, either by continual venipuncture or other nonmedical activities such as frequent drug use. A sample of blood would likely be impossible to obtain from such veins because the flow of blood is not even or regular due to the contortions of the vein. Relatedly, frequent venipuncture may lead to various complications, which may or may not be life threatening, if the venipuncture is not done properly, including bruising, inflammation of the skin and infection. Thus venipuncture must be done properly and efficiently to avoid causing complications or exacerbating any existing conditions.

In addition, blood draw is a very daunting experience for many individuals, even those that are overly familiar with the process. Many individuals are nervous from past venipuncture experiences, which can lead to fainting and the onset of shock during the process. Some people become extremely anxious at the thought of having their blood drawn because they have a fear of some aspect of the procedure, such as a general fear of needles or the fear of being punctured with a needle. An individual that undergoes venipuncture under minimal amounts of stress is much more likely to have a successful blood draw because the unnecessary anxiety and stress can lead to an increased tendency for the vein to collapse. If a method existed by which the location one or more veins could be marked on an individual, venipuncture that is performed for any reason would likely be less stressful because the individual is less fearful. In addition, blood donations would likely increase because a donor would be much more inclined to donate blood if the procedure may seem less intimidating.

Various methodologies and techniques exist in the prior art which are used to mark an area on the body of an individual in connection with a medical procedure to ensure that the procedure is properly done. For example, Williams, et al., U.S. Pat. No. 4,860,331 issued Aug. 22, 1989 discloses a marker device utilized in body scanning, which is adhesively applied to the skin of the patient. The device comprises a flexible adhesive-coated tape which is generally transparent and includes opaque reference lines in addition to biopsy holes that facilitate the insertion of a needle into the skin at a particular location. The device is primarily used in connection with computerized axial tomography (CAT) but it has application to various other scanning techniques including ultra-sound.

The purpose of the device is to provide detectable effects on the scanning waves striking the device during the procedure so that the markings are distinguishable on the image. The reference lines enable the internal components of the body of the individual over which the tape is placed to be located relative to the image of the markings superimposed thereon once the procedure is complete. The biopsy holes further provide a reference point where a needle may be inserted to obtain a sample, if necessary. Williams marks a location relative to the immediate placement of the device causing the reference locations to only be accurate so long as the tape is adhered to the skin. Once the device is removed from the individual, the reference lines and the biopsy holes are no longer accurate unless the device is reaffixed to the individual and the CAT procedure is once again performed.

Heintz, United States Patent Publication No. 2006/0266368 published on Nov. 30, 2006 is directed to a device which is used in medical procedures. Heintz discloses a device that is used to provide a pre-surgical warning on the body of a patient that is about to have surgery on a particular body segment. Specifically, the device provides a pre-surgical safety warning, notification and/or alert to avoid surgical procedures from being accidentally performed on an unintended limb, tissue or other body part of a patient. The pre-surgical warning is provided on a strip of vinyl, PVC, cellulose, fabric or other material and the material may further have a variety of shapes, sizes and configurations. The device warns generally of the particular body part as it can be placed anywhere along the unintended body part. However, it is not used to mark a more specific location on the body of the individual where the procedure should be performed, such as where a cut should be made by a surgeon.

Fishell, et al. U.S. Pat. No. 6,985,771 issued Jan. 10, 2006 discloses a system for detecting an acute myocardial infraction at the earliest possible time. The system disclosed by Fishell involves the administration of a thrombolytic type medication directly into the blood circulation of an individual by means of a pass-through drug port. The drug port employs a septum on the outer surface, the location of which is just under the skin of the individual. The septum is not only detectable by shape and location but also by a tattoo-like mark on the skin. The mark is placed on the skin directly over the center of the septum of the port. The mark designates the exact precise location into which injections should be made. By providing only one access site on the individual, the same location is subject to repeated access which can lead to the complication already discussed, such as thrombosed veins.

There also exist in the prior art several devices for locating veins on an individual for the purpose of performing a diagnostic or therapeutic medical procedure. For example, Canada, U.S. Pat. No. 2,234,961 issued Aug. 5, 1938 discloses a Vein Holder. Canada teaches a tourniquet which includes a readily manipulable device utilized to press down on opposite sides of a vein and hold it against displacement to facilitate insertion of a hypodermic needle and raise the vein. The device is placed about two inches above the vein to be used and has two downward sloping legs which are positioned to press down at a single location on either side of the vein to be punctured. Similarly, DeVitilis, U.S. Pat. No. 4,332,248 for a Medical Apparatus issued on Jun. 1, 1982 discloses an apparatus to aid in inserting the needle of a syringe. The apparatus includes a pair of members that are placed on the sides of a vein to prevent movement of the vein while it is being pierced by the needle. Both of these devices are complicated, bulky and cumbersome to utilize.

Vertenstein, U.S. Pat. No. 5,167,629 issued Dec. 1, 1992 discloses a vein locator for locating a vein of an individual for insertion of a venous access catheter. The locator comprises a sleeve that partially encompasses a vein to be located at a later time and a ring removably attached to the sleeve. The sleeve is sutured to subcutaneous tissues in a fixed location with respect to the skin. The ring has protuberances around an opening so that the protuberances can be felt by palpation of the skin of the individual to locate the vein. The Vertenstein device requires that the device be secured to the vein inside the body. Both placement and removal of the device require a surgical procedure to be performed on the potentially fragile veins of the individual. Further, the device may consist of screw, rivets, clips, cement and the like. If any accidental harm, such as forceful jarring, should occur to the skin, the device may likewise be damaged which may lead to the protrusion into the vein by a damaged portion of the device.

What is lacking in the art is a method of marking a location on an anatomical portion of an individual for venous puncture access to enable a medical professional to quickly locate a vein of an individual when performing any diagnostic or therapeutic procedure.

### BRIEF SUMMARY

A method of marking the boundaries of at least one vessel with a venous access marking for locating a venous puncture access site to facilitate the introduction or withdrawal of fluid from the vessel in connection with a medical procedure is disclosed. The method comprises the steps of: designating an anatomical portion of an individual on which the medical procedure is performed; locating a vessel along the anatomical portion of the individual; applying a marking material to designate the boundaries of the vessel, the marking material defining a region for venous puncture access; cleansing an area proximate the venous access marking and accessing the region of venous puncture access using the venous access marking as a visual reference.

Vessels may include without limitation arteries, arterioles, capillaries, venules and veins, as the method described herein is equally applicable to any type of vessel. The appropriate vessels may include but are not limited to the medial cubital vein, the basilica vein, the cephalic vein, the jugular vein and the saphenous vein. The methodology may be utilized in connection with venipuncture for withdrawing blood or other fluids from the body of an individual. The methodology may also be utilized in connection with the injection of blood or other fluids into the body of an individual.

The method of marking may be performed on an arm, a wrist, a foot, a hand or any other vessel that is readily accessible through the skin. The marking material may be temporary material, such as a water-soluble paint, dye, ink or henna material. The marking material may be a permanent material, such as a tattoo ink. The marking material may only reflect and be visible under ultraviolet or infrared light. The venous access marking may be configured on the user as a round dot, a circle, a square, a rectangle, a triangle, a line or any other geometric figure. The venous access marking may also be configured as a word, a numeral or a pictorial image or any other designation that can assist a phlebotomist in visualizing the location of the vessel to be accessed. The location of the vessel may be determined by exploratory procedures including, without limitation, ultrasound. Exploratory procedures may further be utilized to determine the depth of the vessel below the surface of the skin of the individual and the venous access marking may be configured and applied to reflect such depth. Thus, the word, numeral, pictorial image or other configuration may indicate the depth of the vessel below the surface of the skin of the individual.

A venous access marking is also disclosed for designating the boundaries of at least one vein to facilitate the introduction or withdrawing of fluid from the vein. The venous access marking comprises a marking material applied along the boundaries of a vein, the marking material comprising an arrangement of indicators to designate a region of venous puncture access locatable by visual reference.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates an individual undergoing venipuncture, the individual having a venous access marking to mark the boundaries of a vein to designate a region of venous puncture access.

FIG. 2 represents an embodiment of the venous access marking on the anterior side of the arm of an individual.

FIG. 3 represents another embodiment of the venous access marking on the anterior side of the arm of an individual.

FIG. 4 represents another embodiment of the venous access marking on the anterior side of the arm of an individual.

FIG. 5 represents another embodiment of the venous access marking on the anterior side of the arm of an individual.

FIG. 6 is a flow chart of the methodology of the placement of the venous access marking and the procedure for locating the boundaries of a vein and a region of venous puncture access using the venous access marking as a reference point.

FIG. 7 is represents an embodiment of a marking device that may be used to apply the venous access marking on the arm of an individual.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for utilizing the invention. It is to be understood, however, that the same or equivalent functions and sequences may be accomplished by different embodiments and that they are intended to be encompassed within the scope of the invention.

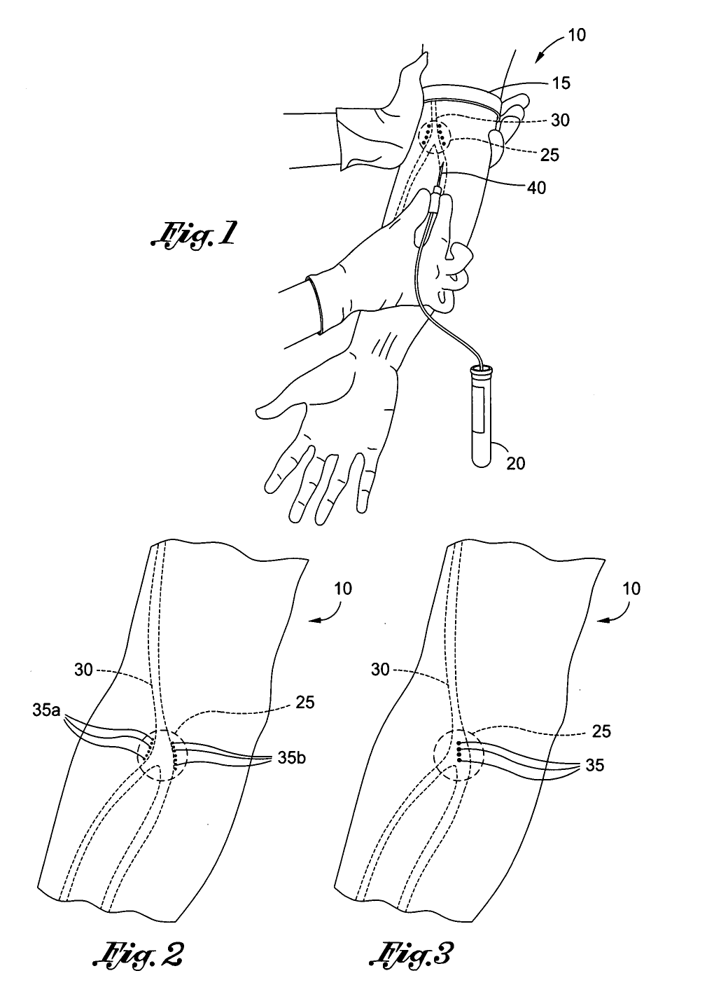

FIG. 1 illustrates a diagram of the inner side of the right arm of an individual, which is generally designated by the numeral 10. A medical professional, such as a phlebotomist, has inserted a needle 40 into a vein 30 of the individual for the purpose of drawing blood, or other fluid, from the vein 30 of the individual in connection with an evacuation tube 20. The individual has a tourniquet 15 around the upper portion of the arm 10. The arm 10 of the individual further includes a venous access marking 25 that enables a medical professional, such as a phlebotomist, to quickly find a locus for access to a vein suitable for venipuncture.

The individual on whom the venous access marking 25 may be made can be any person that is undergoing blood draw. The necessity for the blood draw and the venous access marking 25 may be short term, such as in connection with a hospital stay, or long term, such as in connection with a chronic condition. The venous access marking 25 may be made on a person to facilitate routine blood draws. The venous access marking 25 may also be made on an individual who donates blood with any frequency. It should be noted that the procedure for marking a venous access site and a method of accessing the venous site in connection with the venous access marking 25 as a guideline may also be applied to animals, such as cats, dogs and horses, as well. In addition, the venous access marking 25 is not limited to use in connection with only venipuncture. The venous access marking 25 may also be used in connection with any procedure that can be facilitated by such a marking to indicate the location of a vein that is appropriate for venous puncture access for therapeutic or diagnostic purposes. For example, the venous access marking 25 may be useful for the placement of a catheter line to administer one of the following: a transfusion, fluid, medication, nutrients, or any other diagnostic agents. The venous access marking 25 may be utilized for the entrance into tissue, muscles, vessels which may include veins, arteries, capillaries, venules and arterioles, or any other component of the body.

The material used for the venous access marking 25 may be of a temporary nature or the material may be permanent. For example, an individual may be admitted to the hospital for an extended period of time in connection with a medical procedure. During that stay, the individual may require frequent blood draw. Similarly, an individual may be receiving outpatient treatment at a clinic for a condition that requires blood draw at an increased frequency for a time period of limited duration. In either case, the individual may have a venous access marking 25 applied to assist the drawer in performing venipuncture. The venous access marking 25 may be applied utilizing a material that can be washed or worn off. For example, a temporary venous access marking 25 may be applied using a water-soluble paint, dye or ink. The venous access marking 25 may be easily removed by wetting or rubbing the skin. A henna material may also be utilized to apply the venous access marking 25 in which case the marking would wear off through the natural sloughing of the skin which occurs every three to four weeks.

The venous access marking 25 may also be applied using a material of a more permanent nature. For example, an individual may suffer from a chronic medical condition, such as diabetes or a liver dysfunction, which requires the regular draw of blood from the individual typically over the lifetime of the individual. In this case, the individual may want to have the venous access marking 25 permanently applied so that it does not wash or wear off. Thus, the venous access marking 25 may be applied to the skin of the individual using a permanent ink or dye in connection with a procedure that involves injection into the skin, such as through tattoo technology. Thus, once the ink or dye has been injected into the dermis of the skin of the individual, the ink or dye should generally remain permanently in place.

Regardless of whether the venous access marking 25 is temporary or permanent, the material that is utilized should be a color that contrasts with the skin, with typical colors including black, blue or green. Alternatively, the material may be a special color that only reflects under specialized lighting such as ultraviolet or infrared. For example, the person may believe that the venous access marking 25 is disfiguring, embarrassing or socially undesirable although necessary for their medical well-being. Moreover, some religions prevent an individual from placing a permanent marking, such as a tattoo, on their person. A venous access marking 25 that is only visible under certain conditions would be desirable for any such individual that does not want to have a permanent marking that can always be viewed by others. Thus, when the individual is undergoing a procedure that requires the use of the venous access marking 25, special lighting is required to make the marking 25 visible.

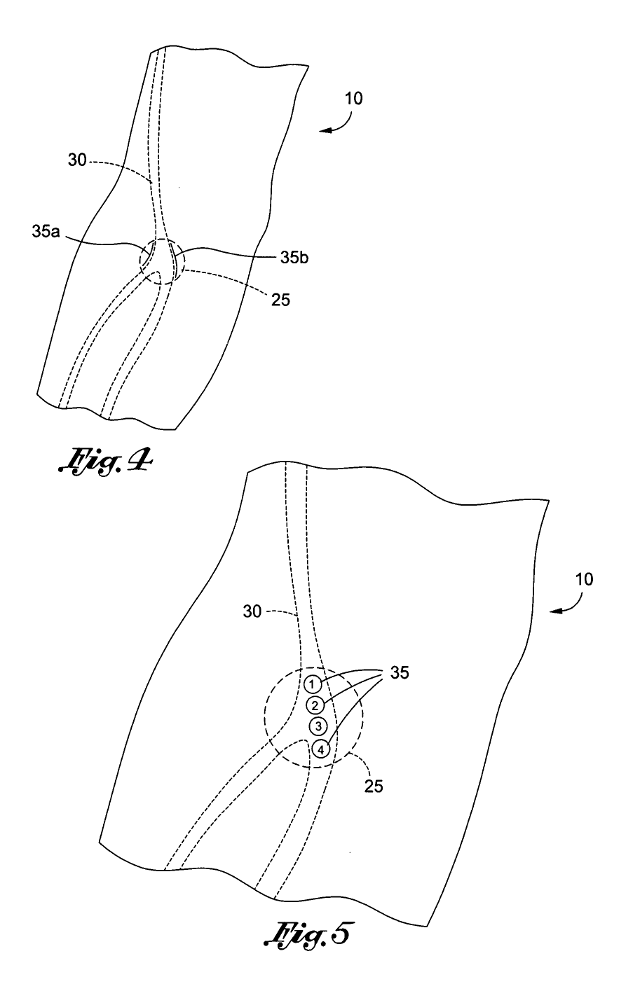

Referring to FIGS. 1 and 2, the configuration of the venous access marking 25 may consist of at least one pair of indicators, 35a, 35b. Each indicator shown in FIG. 2 is generally in the shape of a round dot. It should be noted that each indicator may be any size or configuration that allows the indicator to serve as a guideline in connection with the boundary of a selected vessel, the indicators further defining a region of venous puncture access without interfering with the ability to accurately pinpoint the location of the vein and access the same. Thus, each indicator may have any geometric configuration, including but not limited to a square a circle, a rectangle, a triangle, a line or any other geometric figure. Further each indicator may also be in the form of words, numerals or pictorial images. The numerals may indicate a series of locations which should be accessed in a particular order or the numerals may further indicate a depth of the particular vessel location below the surface of the skin. The indicator may have any configuration to assist a phlebotomist in visualizing the location of the vessel to be accessed.

For example, in FIG. 2, the venous access marking 25 consists of a plurality of indicators 35a, 35b with each indicator 35a, 35b located on either side of the vein 30. Additional pairs of indicators 35a, 35b may be placed on the arm, as necessary to mark the boundary of the vein. The phlebotomist, or any other medical personnel involved in the blood draw, can insert a medical device, including without limitation, a needle, a syringe, a catheter, a probe, or other device, depending on the procedure, within the region of the vein designated by the venous access marking 25.

FIG. 3 represents another embodiment of a venous access marking 25 which consists of a single linear vertical arrangement of indicators 35 with each successive indicator 35 located immediately below the previous indicator. The linear arrangement of indicators 35 is applied so as to be situated along the widest part of the vein.

FIG. 4 represents another embodiment of a venous access marking 25 which comprises a pair of indicators 35a, 35b with each indicator 35 being a line. Once again, the line is applied to mark the boundaries of the vein to be accessed. Each line may be of any length, but should at least be of a length suitable to define a region of access.

Finally, FIG. 5 represents another embodiment of a venous access marking 25 that consists of a single linear vertical arrangement of indicators 35 with each indicator 35 representing a numeral. The indicators 35 are sequentially numbered to provide a guideline to track the last site of venous access. The indicators 35 may also consist of a variety of shapes, words or configurations so long as the indicators serve to mark the last access site.

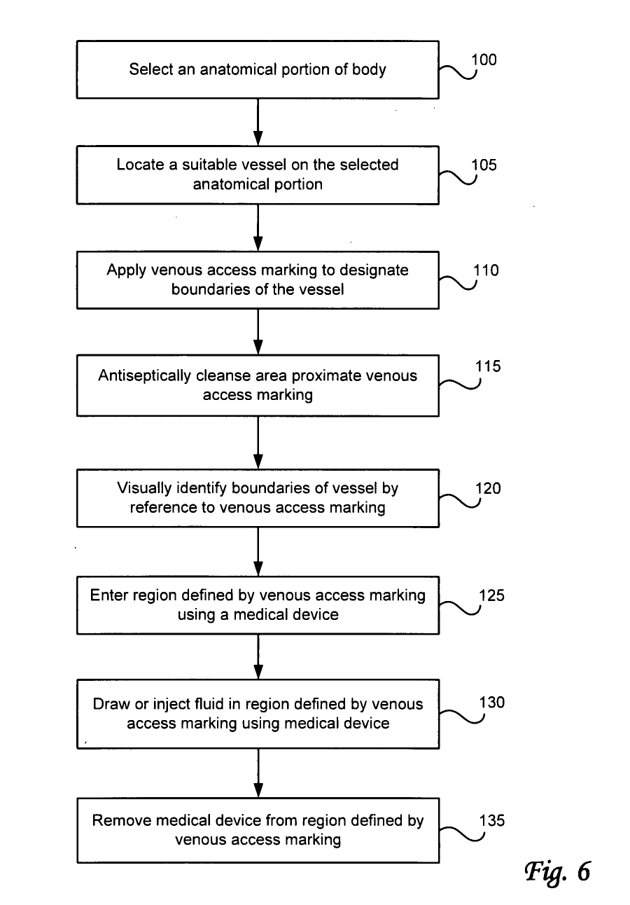

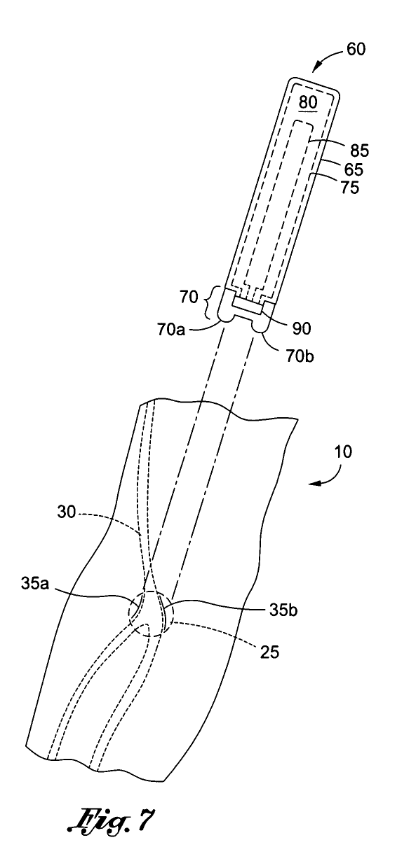

Referring to FIG. 6, the methodology of marking a vessel and performing a procedure utilizing the venous access marking 25 to define the boundaries of the vessel is described. The methodology may be utilized in connection with venipuncture for withdrawing blood or other fluids from the body of an individual. The methodology may also be utilized in connection with the injection of blood or other fluids into the body of an individual. First at step 100, the medical professional must select an anatomical portion of the body of the individual on which a diagnostic or therapeutic procedure will be performed. In the case of venipuncture, the most desirable veins are located either on the arm, the hand, the wrist or depending on the circumstances, the foot. Next, at step 105, the medical professional finds a suitable vein from which to draw blood. The vein may be located by direct palpation. If no vein is immediately palpable, then the individual is required to make a fist, clench and release until a vein is located from which blood can be drawn. The application of an elastic band or other type of tourniquet, as shown in FIG. 1, may also cause a desirable vein in the arm to become visible as the band causes a pressure to be applied to the upper arm to restrict blood flow through the vein. The pressure caused by the band further causes the veins below the elastic band to distend or fill with blood. The location of the vessel may be determined by any exploratory type procedure including, without limitation, ultrasound. The exploratory procedure may further be utilized to determine the depth of the vessel and the venous access marking may be applied accordingly as further described herein.

The most common vein used in venipuncture is the median cubital vein 30, which is shown in FIGS. 1-5. The median cubital vein 30 is located on the anterior forearm which is the side of the arm 10 within the fold of the elbow. In many individuals, the medial cubital vein 30 lies close to the surface of the skin. The proximity of this vein to the surface of the skin provides convenient access which lends to an accurate venous accurate marking 25 being placed on the skin to indicate the location of this vein. Moreover, the anterior forearm also does not have a large nerve supply. Thus, if the venous access marking 25 is placed by method of tattoo injection or any such procedure providing a marking of a more permanent nature, the process will cause only minimal discomfort for the individual. The cephalic and the basilica veins may also be utilized for venipuncture. The dorsal side of the hand or wrist can be used for blood draw and a venous access marking 25 applied in connection therewith if the veins of the arm are determined to be unsuitable. Similarly, certain veins in the foot may also be utilized.

Once the desired vein is located the drawer applies a venous access marking 25 at step 110 to designate the boundaries of the vein which further define a region of venous puncture access. As previously stated, tattoo technology may be utilized to apply a venous access marking of a more permanent nature. However, a venous access marking 25 may be applied utilizing a marker device, such as that shown in FIG. 7, which carries an ink which may also be permanent, semi-permanent or temporary.

For example, a suitable marking device 60 may include a barrel 65 and a tip 70. The barrel 65 is an elongated body which may have a tubular configuration, although one skilled in the art can appreciate that the barrel 65 may have any geometric configuration that facilitates the gripping and manipulation of the marking device 60 to apply a venous access marking 25. The barrel 65 is designed to include at least one ink reservoir 75 within for holding a marking material 80. The ink reservoir 75 may further include a fibrous reservoir member 85 therein to hold the marking material 80 in the interstices between the fibrous members. Barrel 65 preferably includes an aperture 90 at the end proximate the tip 70 to allow the marking material 80 to transfer through for marking. The ink reservoir 75 is attached to the tip 70 through which ink droplets are delivered to the skin of the individual. The marking device 60 may be a channel marker such as that shown in FIG. 6 with the tip 70 having a first portion 70a and a second portion 70b, the first and second portions 70a, 70b being separated by a width that approximates the width of a suitable vessel 30. The marking device 60 is placed in contact with the skin and is moved along the skin to apply the venous access marking 25. It can be appreciated that depending on the type of venous access marking 25 being applied to the skin, the marking device 60 may require a variable type of manipulation, including without limitation a stamping or direct pressure of the skin.

The next step in the procedure may occur immediately following the application of the venous access marking 25 at step 110 or some time may have elapsed. Once the venous access marking 25 has been applied, the area proximate and including the venous access marking is antiseptically cleansed at step 115 using any appropriate antibacterial or antimicrobial agent including soap, alcohol or iodine. At step 120, the site of venous access is identified and located by visual reference to the venous access marking which define the boundaries of the vein and the region of venous puncture access. Thus, the need for additional palpation is eliminated. At step 125, a sterile needle or other medical device is inserted into the vein within the region of venous puncture access. The needle or other medical device is inserted quickly and smoothly through the skin to minimize pain and prevent blood from spouting out, and blood or other fluid is collected in an air-tight vial or a syringe. At step 130, the procedure is performed which may include blood or other fluid being drawn from or injected into the region defined by the venous access markings. At step 135, once the blood or other fluid has been collected or injected, the needle is removed, and the puncture site is covered to stop any bleeding.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of marking the boundaries of at least one vessel with a venous access marking for locating a venous puncture access site to facilitate the introduction or withdrawal of fluid from the vessel in connection with a medical procedure, the method comprising the steps of:
designating an anatomical portion of an individual on which the medical procedure is performed;
locating a vessel along the anatomical portion of the individual;
applying a marking material to designate the boundaries of the vessel, the marking material defining a region for venous puncture access;
cleansing an area proximate the venous access marking and accessing the region of venous puncture access using the venous access marking as a visual reference.

2. The method of marking the boundaries of at least one vessel with a venous access marking for locating a venous puncture access site of claim 1, wherein the anatomical portion is one of an arm, a wrist, a foot and a hand.

3. The method of marking the boundaries of at least one vessel with a venous access marking for locating a venous puncture access site of claim 1, wherein the vessel is one of a vein, an artery, a capillary, a arteriole and a venule.

4. The method of marking the boundaries of at least one vessel with a venous access marking for locating a venous puncture access site of claim 1, wherein the vessel is one of the medial cubital vein, the basilica vein and the cephalic vein.

5. The method of marking the boundaries of at least one vessel with a venous access marking for locating a venous puncture access site of claim 1, wherein the vessel is one of the jugular and the saphenous vein.

6. The method of marking the boundaries of at least one vessel with a venous access marking for locating a venous puncture access site of claim 1, wherein the marking material is temporary.

7. The method of marking the boundaries of at least one vessel with a venous access marking for locating a venous puncture access site claim 4, wherein the marking material is one of a water-soluble paint, dye or ink.

8. The method of marking the boundaries of at least one vessel with a venous access marking for locating a venous puncture access site claim 4, wherein the marking material is henna.

9. The method of marking the boundaries of at least one vessel with a venous access marking for locating a venous puncture access site claim 1, wherein the marking material is permanent.

10. The method of marking the boundaries of at least one vessel with a venous access marking for locating a venous puncture access site claim 1, wherein the marking material reflects ultraviolet light.

11. The method of marking the boundaries of at least one vessel with a venous access marking for locating a venous puncture access site claim 1, wherein the marking material reflects infrared light.

12. The method of marking the boundaries of at least one vessel with a venous access marking for locating a venous puncture access site claim 1, wherein the marking material is configured as one of a round dot, a square, a rectangle, a triangle and a line.

13. The method of marking the boundaries of at least one vessel with a venous access marking for locating a venous puncture access site claim 1, wherein the marking material is configured as one of a word, a numeral and a pictorial image.

14. A venous access marking for designating the boundaries of at least one vessel to facilitate the introduction or withdrawing of fluid from the vessel, the venous access marking comprising:
a marking material applied along the boundaries of a vessel, the marking material comprising an arrangement of indicators to designate a region of venous puncture access locatable by visual reference.

15. The venous access marking of claim 14, wherein the marking material is temporary.

16. The venous access marking of claim 15, wherein the marking material is one of a water-soluble paint, dye or ink.

17. The venous access marking of claim 15, wherein the marking material is henna.

18. The venous access marking of claim 14, wherein the marking material is permanent.

19. The venous access marking of claim 14 wherein the marking material reflects one of ultraviolet light and infrared light.

20. The venous access marking of claim 14 wherein each indicator is one of a round dot, a square, a rectangle, a triangle and a line.

21. The venous access marking of claim 14 wherein each indicator is one of a word, a numeral and a pictorial image.

22. The venous access marking of claim 14, wherein the vessel is the median cubital vein.
